(19) United States
(12) Patent Application Publication
(10) Pub. No.: US 2017/0209893 A1
(43) Pub. Date: Jul. 27, 2017

(54) MISTING DEVICE WITH QUICK RELEASE INTERCHANGEABLE CARTRIDGE

(71) Applicants: John Swallow, Sandy, UT (US); Matthew Hawkes, South Jordan, UT (US)

(72) Inventors: John Swallow, Sandy, UT (US); Matthew Hawkes, South Jordan, UT (US)

(21) Appl. No.: 15/090,501

(22) Filed: Apr. 4, 2016

Related U.S. Application Data
(63) Continuation-in-part of application No. 15/003,434, filed on Jan. 21, 2016.

(51) Int. Cl. B05B 17/06; A61M 11/00; B05B 11/00

(52) U.S. Cl. CPC B05B 17/0676; B05B 11/0002; B05B 11/0054; A61M 11/005; A61M 2205/0294

(57) ABSTRACT

A misting device with quick release interchangeable cartridge is disclosed for atomizing and discharging a composition as a mist for topical application of the composition to the body or diffusing the composition into the ambient air. The cartridge comprises an atomizer and quickly attaches and detaches from a body using a coupling mechanism which may create a friction fit engagement. The atomizer atomizes the composition. A base maintains the body in an upright position for formulation of the composition in the cavity of the cartridge.

### PRIORITY CLAIM

This application is a continuation in part of, and claims priority to, U.S. utility patent application Ser. No. 15/003,434 for John Swallow and Matthew Hawkes for a “Misting Device with Quick Release Interchangeable Cartridge” filed Jan. 21, 2016.

### BACKGROUND

Field of the Invention

This invention relates to a diffusing and misting devices, and more particularly relates to diffusing and misting devices with quick release and/or interchangeable cartridge(s) which contain, atomize, and discharge a composition as a mist for uniform application of the composition to the body, or diffusion into the ambient air for inhalation of the misted or diffused substance.

Description of the Related Art

Generally, atomizers, generators, nebulizers and the like are designed for a wide variety of purposes such as medical, therapeutic, and cosmetic applications on the body. Atomizers often include heating or humidification and the spraying of a liquid-based compositions, such as cosmetics, medicines, oils, paints, fertilizers and other liquids.

It is known that a mist is a phenomenon caused by small droplets of liquid that are suspended in air. The mist is most commonly seen where warm, moist air meets sudden cooling, such as in exhaled air in the winter, or when throwing water onto the hot stove of a sauna. The mist can also be created artificially with aerosol canisters if the humidity and temperature conditions are right. An atomizer can also create a mist.

Typically, a mist generator discharges a mist toward the face or the body for the purpose of treating or protecting the body, and specifically the skin. The mist may have a medical, therapeutic, or cosmetic effect on the skin. One example of such a misting device includes a mist generator in which an atomizer atomizes a liquid-based composition, such as a skin lotion. The generated mist is discharged from an atomizer nozzle. A heating element may also be used to nebulize the composition into the desired mist.

Traditional misters either are not refillable, or contain a reservoir, that while refillable, is inefficient. It is difficult for users to take the time cleaning and refilling the reservoir and refilling a reservoir can lead to accidental spilling of contents, which can damage surrounding items and waste. If the reservoir is not properly cleaned, the new composition is mixed with remnants or residue of the prior substance. If only a portion of the contents of a reservoir or cartridge is utilized when the user desires to diffuse or mist a different substance, often the prior substance is discarded, resulting in waste. These difficulties inherent in the prior art tend to make misters commonly used and useful for only one composition without cleaning or without fully utilizing, even if an earlier changing of the contents would otherwise be desirable. Misters are often discarded, perform less favorably or predictably, are not as efficient in terms of conserving expensive contents and/or not reused as commonly as they might otherwise be if improvements to the art were made and if various contents could be swapped out merely by interchanging cartridges or reservoirs or tanks.

Additionally, in traditional misting devices, the atomizer can become clogged with residue, and different sized atomizers are needed for oils of different viscosities. Thus, conventionally, a misting device can optimally mist or atomize only one specific liquid of a predetermined viscosity for which the size of the atomizer has been customized, making interchange of a plurality of liquids unfeasible as the atomizer is contaminated and not properly sized. Overcoming this problem in the prior art is desirable.

Other proposals have involved misting devices for application of a medicine or oil to the air or body. The problem with these misters is that they do not provide a means for quickly and cleanly interchanging different types of compositions without residue contaminants, and they do not maintain the same composition in the same ratios, nor do they maintain the reservoir or cartridge or tank in an upright position (particularly for a hand-held device) for formulation of the composition and for storage. Even though the above cited misters meet some of the needs of the market, a misting device that is operable with a quick release interchangeable cartridge or reservoir to dispense a composition as a uniform mist on a body or into the air is still desired.

### SUMMARY

From the foregoing discussion, it should be apparent that a need exists for a misting device that works with a quick release interchangeable cartridge to formulate, or contain, and discharge a composition as a uniform mist for application to the body for diffusing into the air for aroma-type or direct application therapies. Accordingly, the present invention has been developed to provide a misting device for dispensing of a composition, the device comprising: a handheld body comprising a rechargeable battery and a depressible triggering mechanism; an interchangeable cartridge comprising a reservoir for housing a composition, the cartridge detachably engagable with the body, the cartridge comprising: a channel for directing the composition from the reservoir to an atomizer; the atomizer comprising one or more piezoelectric inducers for atomizing the composition to a mist in response to a user activating a triggering mechanism, the mist expelled through a misting nozzle, the atomizer affixed to the cartridge; a coupling mechanism, the coupling mechanism configured to detachably couple the cartridge to the body; a charging base, the base configured to join with a closed end of the container, the base further configured to maintain the container and the cartridge in a generally upright disposition while charging the battery.

The cartridge may comprise a generally cylindrical shape. The cartridge may be fabricated from a metal alloy. The coupling mechanism may be configured to form a friction fit relationship between the cartridge and the body in some embodiments.

The outlet of the atomizer may comprise an atomizer nozzle. The trigger mechanism may comprise a depressible switch.

The base may, in some embodiments, comprises an aperture, the aperture of the base configured to receive the closed end of the body.

The device may further comprise a power source, the power source configured to power at least one of the following: a heating or misting element, a pump, and the trigger mechanism.

The device may further comprise a pump, the pump configured to draw the composition from the cavity of the cartridge to the atomizer.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention may be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a top perspective view illustrating one embodiment of a misting device with quick release interchangeable cartridge, in accordance with the present invention;

FIG. 2 is a frontal view of the misting device of FIG. 1, in accordance with the present invention;

FIG. 3 is an elevated side view of the misting device of FIG. 1, in accordance with the present invention;

FIGS. 4A, 4B, and 4C are views of an exemplary cap with an integrated atomizer, where FIG. 4A is a side view, FIG. 4B is a bottom angle perspective view, and FIG. 4C is a bottom view, in accordance with the present invention;

FIG. 5 illustrates a front perspective view of a desktop apparatus with a removable cartridge for misting a composition in accordance with the present invention;

FIG. 6A illustrates a side perspective view of a cap with an integrated atomizer in accordance with the present invention;

FIG. 6B illustrates a top perspective view of a cap with an integrated atomizer in accordance with the present invention;

FIG. 7A is a frontal view of the misting device in accordance with the present invention; and

FIG. 7B is a side view of the misting device in accordance with the present invention.

### DETAILED DESCRIPTION

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

Furthermore, the described features, structures, or characteristics of the invention may be combined in any suitable manner in one or more embodiments. In the following description, numerous specific details are provided, such as examples of programming, software modules, user selections, network transactions, database queries, database structures, hardware modules, hardware circuits, hardware chips, etc., to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

It is an object of the present invention to provide a diffusing and misting device with quick release interchangeable cartridge or reservoir or tank that quickly loads a cartridge into a hand held or stand-alone container (such as what is generally referred to as a diffuser, to formulate, contain, atomize, and discharge a composition as a mist for uniform application of the composition to the body or to the air for inhalation of the misted or diffused substance, and more particularly relates to a misting device that provides a container adapted for hand held or stand-alone operation; whereby the container is defined by an interior chamber adapted for quickly loading and discharging an interchangeable cartridge or reservoir or tank, which may or may not be refillable, containing a composition; whereby the composition, such as medicines and oils, is formulated directly inside the cartridge or is formulated and packaged separately, prior to being inserted into the cartridge or reservoir or tank; whereby the cartridge couples to the interior chamber through a quick release coupling mechanism; whereby the container further includes an inlet tube extending into the interior chamber, an atomizer, an optionally included heating element, and a pump operable through a triggering mechanism to draw the composition through the inlet tube and out of an outlet of the atomizer as a uniform mist or diffused substance; whereby a base supports the container in an upright position for charging the battery and for storage and formulation of the cartridge.

For the purposes of this invention, the terms “cartridge,” “tank” and “reservoir” are synonymous and interchangeable.

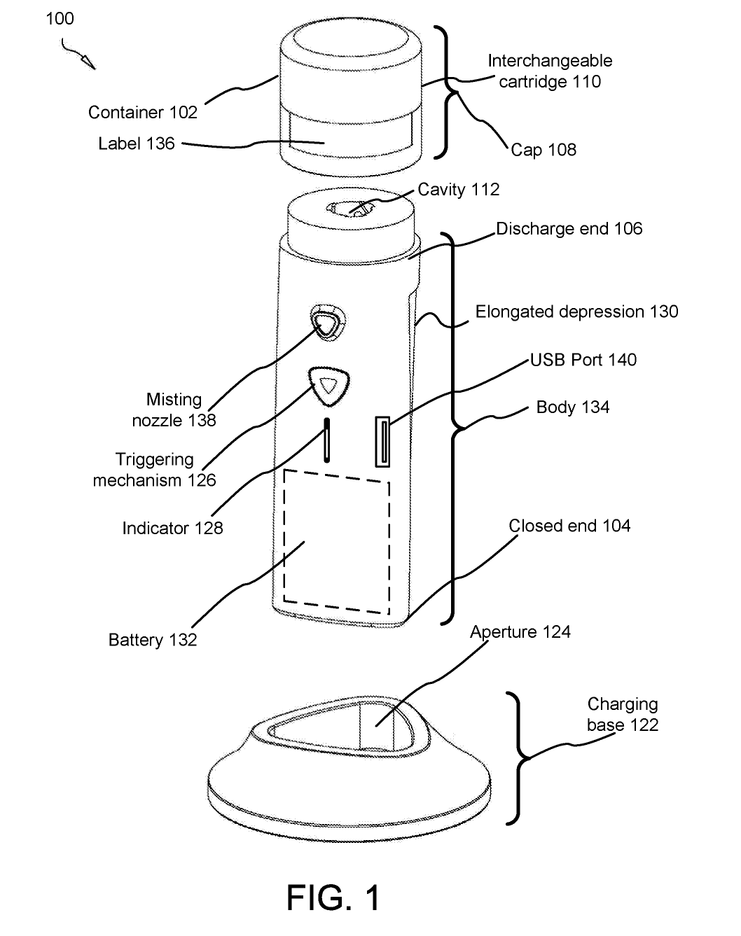

FIG. 1 depicts a misting device or apparatus 100 with a quick release interchangeable cartridge, hereafter, “device 100” that discharges a composition as a uniform mist for application to a body. The composition may include, without limitation, a medicine, an oil, an eye solution, a skin care agent, an essential oil, and a sunscreen composition. The composition may include THC for topical application, vaccinations for inhalation, or the like. The device 100 provides a container 102 that works with a quick release interchangeable cartridge 110 to carry or formulate, contain, and discharge a composition as a uniform mist. In some embodiments, this cartridge 110 may be refillable, and may comprise means known to those of skill in the art for refilling a cartridge, including a screw-down cap, a stopper, a valve, and the like.

The composition may be formulated directly in an interchangeable cartridge or may be pre-formulated and filled 110 and then quickly loaded into an interior chamber of a container 102 for one-handed dispensing. The composition is atomized to enable formation of the uniform mist. A base 122 maintains the container 102 in an upright disposition for enabling formulation of the composition while in the interchangeable cartridge 110 as well as for storage of the device and charging of the battery.

The cartridge 100 in the shown embodiment is the cap, which snaps or screws onto the body 134 of the device 100 as shown. In various embodiments, the cap 108 and/or cartridge 110 comprise a container 102 containing the composition for misting, atomizing, diffusing and/or applying topically to a user's skin. The atomized composition may be inhaled.

The cap 108 may comprise a label 136 or advertisement printed, adhered, engraved or otherwise displayed on the cap 108. The label 136 may comprise a two dimensional trademark, brand name, or identification of the composition in the container 102.

The efficacy of the composition may be enhanced through heating or another form of atomizing and uniform dispersion as a mist. Thus, the device 100 enables: facilitated storage and portability of the composition in a hand held container 102; formulation of the composition directly in an interchangeable cartridge 110 that quickly loads and unloads in the container 102; secure disposition of the composition in an upright position during formulation and while being stored; and atomization of the composition through heating and/or passing through a piezoelectric plate to create a uniform discharge of a mist on an affected body. The cartridge or reservoir or tank could just as easily be utilized in a stand-alone device that is not hand-held.

The body 134 of the device 100 comprises a discharge end 106, which engages the cap 108, and a closed end 104.

A triggering mechanism 126, or depressible button, is triggered by a user to activate the device 100, which expels atomized fluid from the misting nozzle 138.

In various embodiments, the body 134 comprises an internal battery 132, which is charged by the charging base 122. The charging base 122 is connected, in various embodiments, to a standard 120 V A/C power outlet. The body 134 may also comprise a USB Port 140 for charging portable devices, including mobile phones.

The device 100 may also comprise one or more indicators 128 for displaying either the amount of power left in the battery 132 or the fluid left in the container 102.

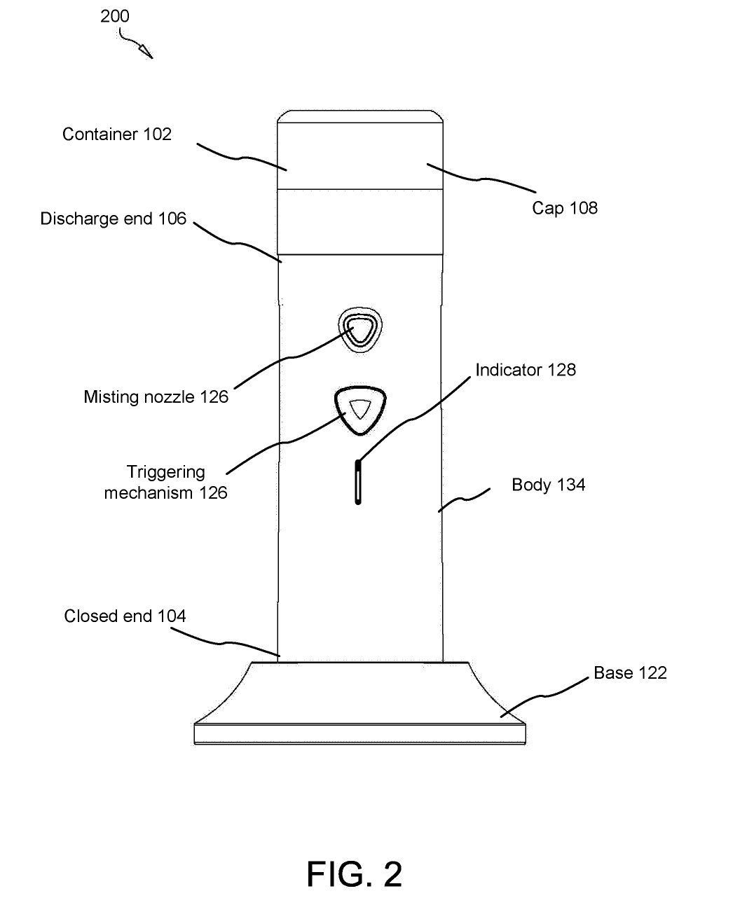

As illustrated in FIG. 2, the device 100 comprises a container 102 adapted for hand held operation and uniform dispensing of the composition. In one embodiment, the container 102 comprises a generally cylindrical shape. Though in other embodiments, the container 102 may include, without limitation, a rectangular shape, a cubicle shape, and a spherical shape. In one embodiment, an elongated depression 130 runs along the length of the container 102. The elongated depression 130 provides a gripping surface for enhanced manipulation of the container 102. The container 102 is defined by an interior chamber that is adapted for quickly loading and discharging an interchangeable cartridge 110 of a composition.

Various containers 102 may be interchanged with the device 200, comprising various species of compositions. In some embodiments, the container 102 is refillable. In other embodiments, the container 102 is pressurized. In other embodiments, the container 102 is prefilled and is neither pressurized nor refillable.

In some embodiments, the container 102 forms part of the cap 108. In other embodiments, the container 102 is a cartridge or reservoir or tank detachably inserted into or onto the body 134.

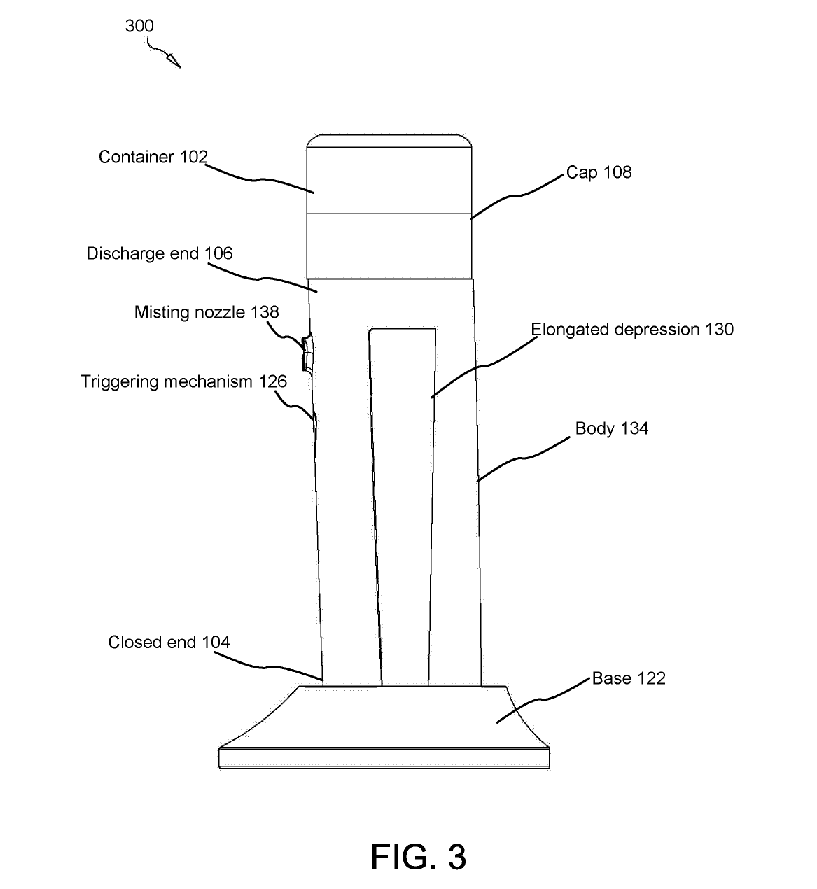

As referenced in FIG. 3, the container 102 may also include a discharge end 106 and a closed end 104. The discharge end 106 is open and enables passage of the interchangeable cartridge 110. A cap 108 may detachably attach to the discharge end 106. In one embodiment, the cap 108 comprises a plurality of cap sidewalls 114. The cap sidewalls 114 are shaped and dimensioned to form a snug fit with the discharge end 106 of the container 102. Suitable materials for the container 102 may include, without limitation, aluminum, steel, metal alloys, and a rigid polymer.

In some embodiments, the interior chamber of the container 102 may include a coupling mechanism (not shown) that is configured to enable detachable attachment between the cartridge 110 and the interior compartment of the container 102. The coupling mechanism enables quick loading and unloading of the interchangeable cartridge 110 in the interior compartment. In one embodiment, the coupling mechanism forms a friction fit engagement between an outer surface of the interchangeable cartridge 110 and the surface of the inner compartment. In one possible embodiment, the coupling mechanism is a snap-fit connection. In another embodiment, the coupling mechanism is a pressurized twist connection having threaded surfaces on the outside surface of the cavity 112 and the discharge end 106 of the container 102. In yet another embodiment, the coupling mechanism enables the interchangeable cartridge 110 to clip in and out of the interior chamber through one-handed manipulation.

Looking back at FIG. 1, the interchangeable cartridge 110 is defined by a cavity 112 and a cartridge opening (not shown). The cavity 112 is sized and dimensioned to enable formulation of the composition directly inside the interchangeable cartridge 110. The cartridge opening forms an inlet for combining ingredients of the composition for formulation, and subsequently discharging the composition.

In some embodiments, the composition may be formulated in the interchangeable cartridge 110 prior to coupling in the interior chamber of the container 102. In other embodiments, the composition may be formulated in the interchangeable cartridge 110 while coupled to the interior chamber of the container 102. In this manner, the varieties of compositions may be quickly adapted, changed, and discharged as a mist for appropriate application on the body.

For example, without limitation, an eye drop solution may be formulated and mixed in a first interchangeable cartridge, and then quickly loaded into the container 102 for application to the eye in the form of a mist. A second interchangeable cartridge containing a sunscreen lotion may then be formulated directly in a second cartridge outside the container 102. The first interchangeable cartridge may then decouple from the container 102 through the coupling mechanism. The second interchangeable cartridge may then replace the first cartridge by quickly loading into the interior chamber. The sunscreen lotion may then be applied as a uniform mist. Furthermore, additional compositions may be formulated in the first and second interchangeable cartridges after thorough cleaning of their respective cavities. Although not shown, multiple cartridges, reservoirs or tanks may be prefilled and simply interchanged without the need for the user to formulate or fill any of the cartridges or reservoirs or tanks, and simply dispose of the cartridge after the contents are emptied or interchanged and save the partially-used cartridge at any time.

In some embodiments, a base 122 supports the container 102 in a generally upright orientation. The base 122 couples to the closed end 104 of the container 102, such that the discharge end 106 is free to dispense the composition. In one embodiment, the base 122 comprises an aperture 124 that is sized and dimensioned to receive the closed end 104 of the container 102. This upright orientation of the container 102 allows the composition to be formulated directly in the cartridge 110 while loaded in the container 102. This upright disposition helps reduce undesirable settling and separation of the composition.

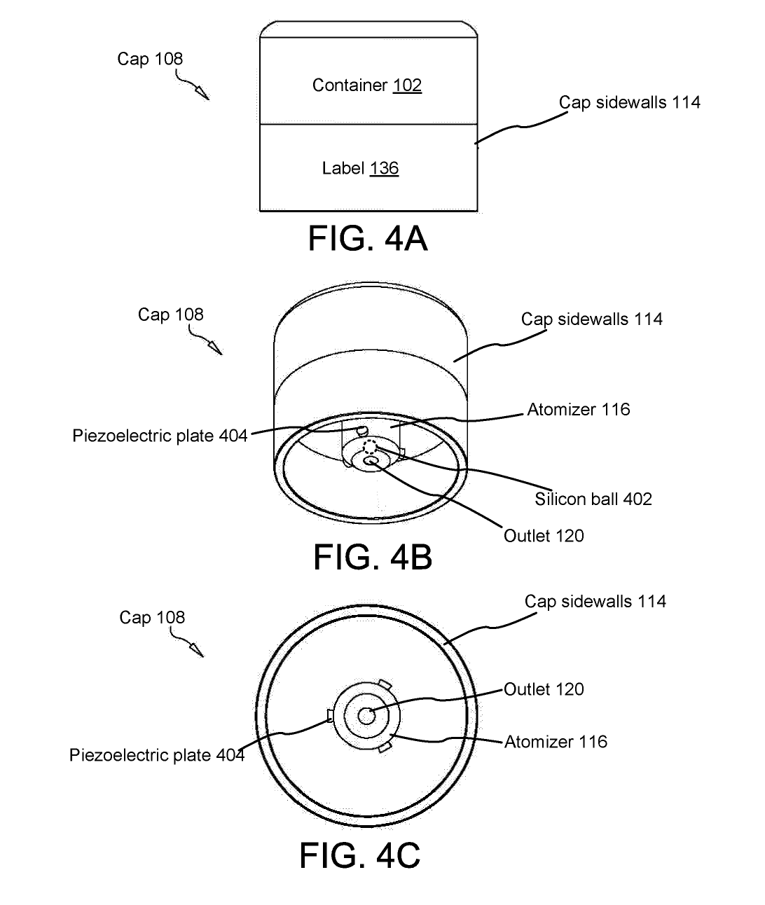

Looking now at FIGS. 4A and 4B, the device 100 further includes an atomizer 116. The atomizer 116 is configured to atomize the composition into the mist. The atomizer 116 is disposed at the discharge end 106 of the container 102. In one embodiment, the atomizer 116 integrates into the cap 108. Though in other embodiments, the atomizer 116 detachably attaches to the discharge end 106 of the interior chamber.

In various embodiments, the cap 108 comprises a cylinder with an open lower surface which is interiorly threaded for engaging a corresponding exteriorly-threaded surface on the body 134 and/or discharge end 106 of the device 100, 200, 300.

The atomizer 116 may comprise a silicon ball 402 which slows flow of atomized fluid through the outlet 120. Fluid expelled through the atomizer is routed through an interior chamber out of the misting nozzle 138 in various embodiments.

In various embodiments, the atomizer 116 comprises an ultrasonic nozzle having one or more piezoelectric plates or transducers 404 (including piezoelectric ceramic elements) for atomizing fluid in the container 102. In various embodiments, the droplets are produced by vibration in the range of 10 to 200 kHz.

The piezoelectric plates 404 may be clad with stainless steel for erosion resistance, and may produce fine and consistent particles sizes of 3 u.m. or less.

The shown embodiment comprises three piezoelectric plates 404, but various other embodiment may include as low as one or as many as hundreds of piezoelectric plates 404.

In some embodiments, the atomizer 116 may include a heating element. In the preferred embodiment, the atomizer comprises a piezoelectric plate.

In some embodiments, an inlet tube (not shown) extends between the atomizer 116 and the interior chamber. The inlet tube carries the composition from the cavity 112 of the interchangeable cartridge 110 to the outlet 120 of the atomizer 116. The device 100 may further include a pump that is operable through a trigger mechanism 126. The pump is configured to draw the composition through the inlet tube and out of an outlet 120 of the atomizer 116 as a mist. In one embodiment, the pump is a simple mechanical pump that creates a vacuum through operation of the trigger mechanism 126. In another embodiment, the pump is electrical.

As shown in FIG. 4C, the atomizer 116 comprises an outlet 120. The atomizer 116 discharges the composition as a uniform mist through the outlet 120. In one embodiment, the outlet 120 may be an atomizer nozzle that produces a fine spray of the composition based on the Venturi effect. Those skilled in the art will recognize that the atomizing function of the device 100 enables the device 100 to be used as a humidifier, a mister, or diffuser. The type of atomization and discharge is at least partially based on adjustments to the type of atomizer 116, and the shape and size of an outlet 120 on the atomizer 116.

As FIG. 3 illustrates, manipulation of a trigger mechanism 126 actuates the pump. The trigger mechanism 126 may be either manually-operable or electrically operable. The trigger mechanism 126 may partially protrude from the side of the container 102 for facilitated manipulation. In one embodiment, the trigger mechanism 126 is a switch that is disposed centrally along the length of the container 102 and operatively connected to the pump. The switch enables selective dispensing of the composition, and is adapted to be operated with one hand.

In another embodiment, an indicator portion 128 operatively attaches to a power source and the trigger mechanism 126. The indicator portion 128 may indicate, through illumination or audible signals, when the power source is depleted or when the trigger mechanism 126 is depressed.

In conclusion, the device 100 comprises substantially, a container 102, an interchangeable cartridge 110, and an atomizer 116. The container 102 is configured to work with the interchangeable cartridge 110. The interchangeable cartridge 110 quickly loads and unloads into the interior chamber of the container 102 through a quick release coupling mechanism. An atomizer 116 atomizes eclectic types of compositions in the form of a mist for uniform application to the body. A base 122 maintains the container 102 in an upright position to enable formulation of the composition directly in the cavity 112 of the cartridge 110, and also to enable upright disposition of the composition during storage.

The misting device 100 may be configured to: store and carry the composition in a hand held container 102; formulate the composition directly in a refillable, interchangeable cartridge 110 that quickly loads and unloads in the container 102; maintain the composition in an upright position during formulation and during storage; and atomize the composition using a piezoelectric plate to create a uniform discharge, which enhances the efficacy of the composition.

In various embodiments, the piezoelectric plates 404 interlock with corresponding recesses on the body 134, these interlocking components forming a quick release mechanism (the corresponding recesses circumscribed the cavity 112). In other embodiments, the cap 108 is threaded interiorly or exteriorly to engage the body 134, this forming the quick release mechanism. In other embodiments, the cap 108 snaps onto the body 134. In still other embodiments, the container 102 in twisted or inserted into a cavity formed by the body 134 for receiving the container 102.

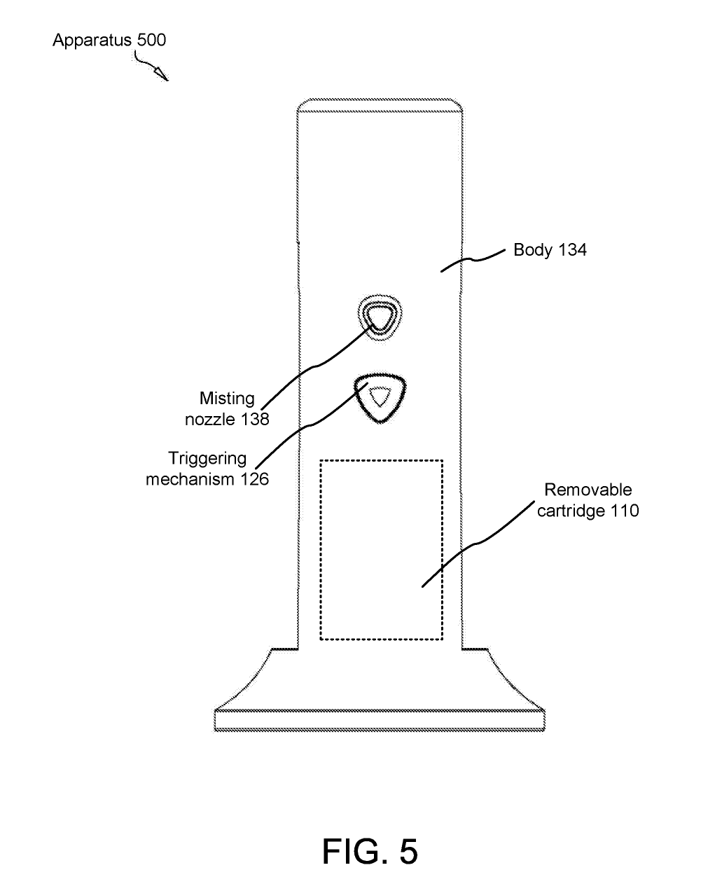

FIG. 5 illustrates a front perspective view of a desktop apparatus 500 with a removable cartridge for misting a composition in accordance with the present invention. The shown device differs from other shown embodiments above inasmuch as the apparatus 500 is a desktop version of the handheld device shown and described in relation to FIGS. 1-4B. The shown embodiment comprises a removable (i.e., interchangeable) cartridge within the body 134 of the apparatus 500. This cartridge 110 may be cubic, cylindrical, or any other regular or irregular shape. The cartridge 110 may be housed anywhere within the body 134 of the device 500.

The apparatus 500 illustrates a device with is meant to stand alone on a table, desk or planar surface. The apparatus 500 may be activated in the same manner as other shown devices, but may lack a cap 108 and/or battery 132.

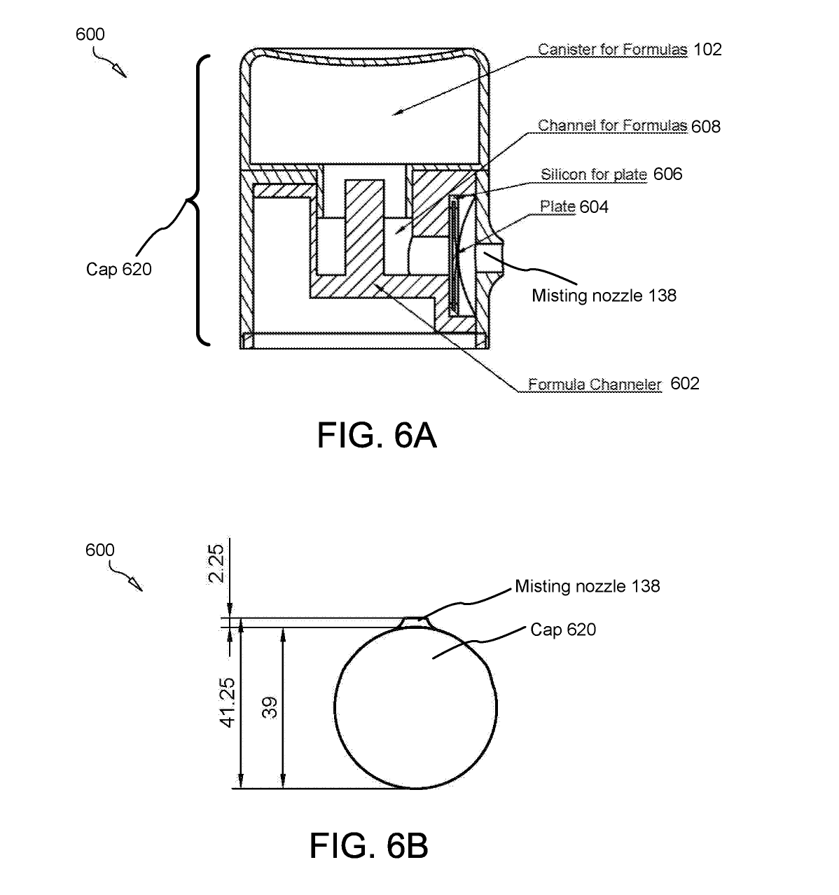

FIG. 6A illustrates a side perspective view of a cap with an integrated atomizer in accordance with the present invention. In the shown embodiment, the cap 620 comprises a container 102 (or canister for formulas) which comprises a formula channeler 602. The formula channeler 602 and the cap 620 form a channel for formulas 608 for channeling the liquid in the container 102 through the atomizer to the misting nozzle 138. The cap 620 comprises silicon 606 for securing one or more a piezoelectric plates 604.

This innovation overcomes many difficulties inherent in the prior art. As the size of the piezoelectric plate 604 must vary with the viscosity of the liquid in the container 102, by incorporating the atomizer into the cap 620 or disposable cartridge rather than the body 134, the present invention allows inclusion of atomizers customized for the predetermined viscosity of the liquid contained in the detachable cap 620 or disposable cartridge.

Additionally, residue builds up over time on the piezoelectric plate 604 of traditional misting devices. Were the atomizer to be included in the body 134 of the apparatus 600, the body itself would have to replaced often as the atomizer became soiled.

FIG. 6B illustrates a top perspective view of a cap 620 with a disposable or interchangeable integrated atomizer in accordance with the present invention. The misting nozzle 138 protrudes from the cap 620.

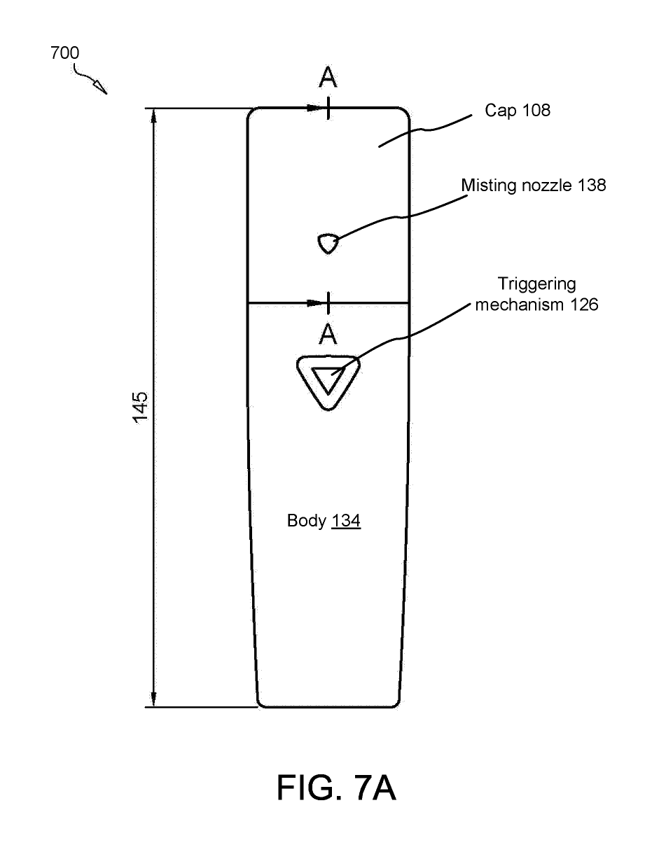

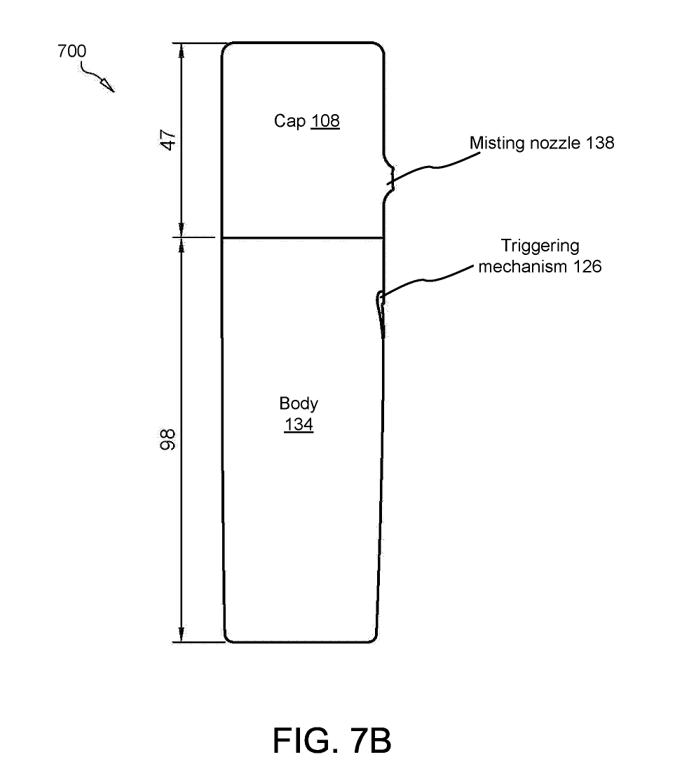

FIG. 7A is a frontal view of the misting device in accordance with the present invention. As shown, the atomized liquid is dispelled from the misting nozzle 138, which nozzle is part of the cap 620. The cap 620 comprises the atomizer, liquid, and piezoelectric plate 604. The body 134 comprises the power supply and triggering mechanism 126 for activating the atomizer. The atomizer comprises the plate 604.

In various embodiments, the cap 620 comprises one or more conductive elements which engage (or come in contact with) one or more corresponding conductive elements in the body 134. When the cap 620 in attached to the body 134, there is electrical conductivity between the triggering mechanism 126 and the atomizer.

FIG. 7B is a side view of the misting device in accordance with the present invention. The triggering mechanism 126 is activated by depression. The liquid is misted/atomized and dispelled through the misting nozzle 138 on the cap 620. The atomizer is in logical connectivity with the triggering mechanism and/or a power supply such as a battery 132.

In various other embodiments of the present invention, the cap, cartridge and/or body of the device 100 may comprise circuitry which is configurable to adjust the width of the misting nozzle. The circuitry may include computer readable memory which stores historical data about user usage of the device, including which cartridge is used, how long it has been used, and how much liquid is remaining in the cartridge. The historical data may be analyzed by a DBMS (database management system) and historical reports displayed graphically. The historical data may also be used to automatically order replacement cartridges for the device 100, and other purposes.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A misting device for dispensing of a composition, the device comprising:
a handheld body comprising a rechargeable battery and a depressible triggering mechanism;
an interchangeable cartridge comprising a reservoir for housing a composition, the cartridge detachably engagable with the body, the cartridge comprising:
a channel for directing the composition from the reservoir to an atomizer;
the atomizer comprising one or more piezoelectric inducers for atomizing the composition to a mist in response to a user activating a triggering mechanism, the mist expelled through a misting nozzle, the atomizer affixed to the cartridge;
a coupling mechanism, the coupling mechanism configured to detachably couple the cartridge to the body;
a charging base, the base configured to join with a closed end of the container, the base further configured to maintain the container and the cartridge in a generally upright disposition while charging the battery.

2. The device of claim 1, wherein the cartridge comprises a generally cylindrical shape.

3. The device of claim 1, wherein the cartridge is fabricated from a metal alloy.

4. The device of claim 1, wherein the coupling mechanism is configured to form a friction fit relationship between the cartridge and the body.

5. The device of claim 1, wherein the outlet of the atomizer comprises an atomizer nozzle.

6. The device of claim 1, wherein the trigger mechanism comprises a depressible switch.

7. The device of claim 1, wherein the base comprises an aperture, the aperture of the base configured to receive the closed end of the body.

8. The device of claim 1, further comprises a power source, the power source configured to power at least one of the following: a heating element, a pump, a piezoelectric plate, and the trigger mechanism.

9. The device of claim 1, further including a pump, the pump configured to draw the composition from the cavity of the cartridge to the atomizer.
